(19) United States
(12) Patent Application Publication
(10) Pub. No.: US 2021/0113841 A1
(43) Pub. Date: Apr. 22, 2021

(54) Compositions and Methods for Treatment of Post-Traumatic Stress Disorder using Closed-Loop Neuromodulation

(71) Applicant: THE REGENTS OF THE UNIVERSITY OF CALIFORNIA, Oakland, CA (US)

(72) Inventors: Jean-Philippe Langevin, Los Angeles, CA (US); Nanthia Suthana, Playa Vista, CA (US)

(21) Appl. No.: 17/077,076

(22) Filed: Oct. 22, 2020

Related U.S. Application Data
(60) Provisional application No. 62/924,423, filed on Oct. 22, 2019.

(51) Int. Cl. A61N 1/36; A61N 1/05

(52) U.S. Cl. CPC A61N 1/36096; A61N 1/36189; A61N 1/36089; A61N 1/0534

(57) ABSTRACT

Provided are compositions and methods for detecting and modulating oscillatory patterns within the basolateral amygdala (BLA) for diagnosis and treatment of anxiety related disorders.

### CROSS REFERENCE TO RELATED APPLICATIONS

This application claims priority to U.S. Provisional Application No. 62/924,423, filed Oct. 22, 2019 which is hereby incorporated by reference herein in its entirety.

### STATEMENT REGARDING FEDERALLY SPONSORED RESEARCH OR DEVELOPMENT

This invention was made with government support under Grant Numbers NS107673, NS103802, awarded by the National Institutes of Health. The government has certain rights in the invention.

### BACKGROUND OF THE INVENTION

Post-traumatic stress disorder (PTSD) is marked by failure of fear extinction and the persistence of enhanced fear state. Hypervigilance, a critical feature of fear state, is characterized by increased alertness, heightened sensory sensitivity and hyperarousal. Understanding the neurophysiology of enhanced fear state and hypervigilance is clinically relevant to develop strategies directly targeting core PTSD symptoms. Functional neuroimaging studies have linked these symptoms to the metabolic reactivity of the basolateral amygdala (BLA) to trauma reminders. In the rodent BLA, fear cells and extinction cells alter their firing rate based on the nature of a stimulus and the influence from the medial prefrontal cortex (mPFC) and ventral hippocampus (vHPC). Together, it is hypothesized that the BLA, mPFC, and vHPC form an anxiety-processing network where the BLA links stimulus to emotion, the vHPC provides memory context, and the mPFC coordinates fear and extinction. However, the exact neural mechanisms modulating the activity of fear and extinction in the BLA of PTSD patients are unknown.

Based on animal studies, there is growing evidence that different neuronal oscillations, and the interactions between them, such as theta-gamma phase amplitude coupling (PAC) are critical in regulating fear processing to initiate an enhanced fear state. In particular, the origin of the dominant theta rhythm modulating the gamma activity in the network appears to predict fear or extinction. For example, if the amplitude of the BLA gamma oscillations is phase-locked to the mPFC theta rhythm, extinction is likely to occur in response to a stimulus. In contrast, if the network-dominant theta rhythm originates from the BLA, fear and hypervigilance are the likely responses to a stimulus (Stujenske et al. 2014, Neuron, 83: 919-933). Additionally, it has been demonstrated that the BLA theta power increases after an aversive stimulus presentation—a signal described as the “neural signature” of enhanced fear state. This BLA theta power increase is thought to prepare the organism for an expected noxious event by enhancing sensory vigilance and alertness (Pare and Collins, 2000, Journal of the Society for Neuroscience 20 (7): 2701-2710).

The investigations of neural correlates of a fear state in humans, barring a few reports, remain largely unexplored. It has been demonstrated that in epilepsy patients, exposure to fearful facial expressions leads to early event-related potentials in the amygdala, which can also be observed—with a delay—in other brain areas, such as the orbitofrontal cortex (OFC)(Krolak-Salmon et al. 2004, Neuron, 42: 665-676). Additionally, the amygdalar-hippocampal dynamics have been implicated in the processing of fearful faces (Zheng et al. 2017, Nature Communications, 8: 14413). Imaging studies in PTSD patient populations have revealed the crucial roles of amygdala and PFC (and the interactions between them), e.g., heightened activity in the right amygdala and marked reduction in mPFC activity (Williams et al. 2006, NeuroImage, 29: 347-357; Stevens et al. 2017, Biological Psychiatry, 81: 1023-1029; Shin, Rauch, and Pitman 2006, Annals of the New York Academy of Sciences, 1071:67-79; Shin et al. 2005, Archives of General Psychiatry 62: 273-281). Nonetheless, neither the interplay between oscillatory properties of brain regions implicated in fear, nor the effect of PTSD on mechanisms of fear have been characterized in humans—both of which play a critical role for potential therapeutic approaches in the PTSD patient population.

Thus, there remains a need in the art for methods to detect and for therapies to treat anxiety related diseases and disorders. The present invention addresses this unmet need.

### SUMMARY OF THE INVENTION

In one embodiment, the invention relates to a method of diagnosing a subject as having an anxiety related disease or disorder, the method comprising administering a stimulus to the subject, detecting at least one oscillatory pattern within the basolateral amygdala (BLA) that is consistent with an anxiety disorder and diagnosing the subject as having an anxiety related disease or disorder based on the detected oscillatory pattern.

In one embodiment, the oscillatory pattern is a pattern of BLA gamma oscillations, a pattern of BLA theta oscillations or a theta-gamma coupling pattern. In one embodiment, the oscillatory pattern is an increase in BLA theta power. In one embodiment, the oscillatory pattern is an increase in BLA gamma power.

In one embodiment, the oscillatory pattern is phase-amplitude coupling between BLA and another relevant brain region. In one embodiment, the relevant brain region is the hippocampus or the orbitofrontal cortex (OFC).

In one embodiment, the oscillatory pattern is a signal detectable by a current closed-loop neuromodulation system.

In one embodiment, the stimulus is a negative neutral stimulus.

In one embodiment, the anxiety disease or disorder is selected from the group consisting of phobias, panic disorders, psychosocial stress (e.g. as seen in disaster, catastrophe or violence victims), obsessive-compulsive disorder, substance use/abuse disorders, addiction, mood disorders (including depression), chronic pain disorders, pervasive development disorders (e.g., autism and intractable aggressive disorder), generalized anxiety disorder and post-traumatic stress disorder (PTSD).

In one embodiment, the invention relates to a method of treating an anxiety related disease or disorder, the method comprising administering brain stimulation to a subject identified as having an anxiety related disease or disorder.

In one embodiment, the brain stimulation alters at least one oscillatory pattern within the BLA. In one embodiment, the oscillatory pattern is selected from the group consisting of a pattern of BLA gamma oscillations and a pattern of BLA theta oscillations and a theta-gamma coupling pattern. In one embodiment, the oscillatory pattern is an increase in BLA theta power. In one embodiment, the oscillatory pattern is an increase in BLA gamma power.

In one embodiment, the oscillatory pattern is phase-amplitude coupling between BLA and another relevant brain region. In one embodiment, the relevant brain region is selected from the group consisting of the hippocampus and the orbitofrontal cortex (OFC).

In one embodiment, the brain stimulation is administered using a neuro-stimulation device. In one embodiment, the neuro-stimulation device comprises an implanted module.

In one embodiment, the brain stimulation is deep brain stimulation.

In one embodiment, the anxiety disease or disorder is selected from the group consisting of phobias, panic disorders, psychosocial stress (e.g. as seen in disaster, catastrophe or violence victims), obsessive-compulsive disorder, substance use/abuse disorders, addiction, mood disorders (including depression), chronic pain disorders, pervasive development disorders (e.g., autism and intractable aggressive disorder), generalized anxiety disorder and post-traumatic stress disorder (PTSD).

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following detailed description of embodiments of the invention will be better understood when read in conjunction with the appended drawings. It should be understood that the invention is not limited to the precise arrangements and instrumentalities of the embodiments shown in the drawings.

FIG. 1A through FIG. 1D depict intracranial recordings in a PTSD patient during IAPS image presentation task. FIG. 1A depicts an example recording electrode located in the right BLA. FIG. 1B depicts an example recording electrode in the right anterior HPC. FIG. 1C depicts an example recording electrode located in the right OFC. FIG. 1D depicts a schematic of the behavioral task. Images were selected from the IAPS data based and included positive, neutral and negative emotions. Image presentation was followed by a prompt that asked the patient to rate the images on a scale of 1-9 in three categories of valence, arousal and dominance.

FIG. 2A through FIG. 2D depict BLA theta power exhibits a sustained increase after the presentation of negative stimuli. FIG. 2A depicts the total theta (3-12 Hz) power as a function of time was significantly higher for negative images (red) compared to positive images (gray) during the time interval 0.70-0.88s after the image onset (t=0) as indicated by the black horizontal line (p<0.05; two-sided t-test; Npositive=20, Nnegative=7; data from 3 images were discarded due to noise). FIG. 2B depicts the same data as in FIG. 2A but in the right HPC. There was no significant difference in theta power in response to negative versus positive images at any time. FIG. 2C depicts the total power during 1s after image onset revealed the difference in power in response to negative and positive images is mostly concentrated around 6 Hz. FIG. 2D depicts the same data as in FIG. 2C, but in the right hippocampus. Shown are mean±s.e.m.

FIG. 3A through FIG. 3D depict different patterns of cross-frequency coupling associated with negative stimuli. FIG. 3A depicts the averaged comodulogram of theta-gamma oscillations within the BLA for negative images (N=7). FIG. 3B depicts the normalized modulation index for PAC between the BLA theta and hippocampal gamma oscillations suggesting that slow gamma is locked to the phase of theta (left). There was no PAC between hippocampal theta and BLA gamma, suggesting that the nature of the interaction between these two regions is directional (right). FIG. 3C depicts a schematic of the phase of theta oscillations. FIG. 3D depicts circular distribution of PAC angles (coupling between the BLA theta and hippocampal gamma) for negative (red; significantly towards 90 degrees; circular V-test, p<1⁻³) and positive (gray) images.

FIG. 4A and FIG. 4B depict decreased OFC theta power after the presentation of negative stimuli. FIG. 4A depicts the total theta power in the OFC was significantly lower 0.86-1.14s after the presentation (t=0s) of negative stimuli (red) compared to positive stimuli (gray) as indicated by the black horizontal line (p<0.05; two-sided t-test; Nₚₒₛᵢₜᵢᵥₑ=20, N_{negative}=7). FIG. 4B depicts the total power during is after image the onset for negative (red) and positive (gray) stimuli. Shown are mean±s.e.m.

FIG. 5A through FIG. 5F depict decreased similar patterns of increased theta power in GAD but not in epilepsy patients. FIG. 5A depicts the average spectrogram in the theta range for negative (left, red) and positive (right, blue) images (left). Normalized difference of the spectrograms for negative and positive images (range: [−1 1]) (right). FIG. 5B depicts the total theta (3-12 Hz) power as a function of time was significantly higher for negative images (red) compared to positive images (blue) during the time interval 0.60-1.10s after the image onset. FIG. 5C and FIG. 5D depict the same analysis as in FIG. 5A and FIG. 5B but in participant with GAD. FIG. 5E and FIG. 5F depict the same analysis as in FIG. 5A and FIG. 5B but in participant with epilepsy.

FIG. 6 depicts that Theta-gamma coupling within the BLA exhibit different patterns for negative versus positive stimuli. High gamma oscillations lock to different phases of the local theta oscillation for negative (left) and positive (right) images.

FIG. 7 depicts exemplary experimental results demonstrating that basolateral amydala theta & gamma activity increases during viewing of negative stimuli.

### DETAILED DESCRIPTION

The present invention relates to compositions and methods for treating neurological and cognitive diseases and disorders. In various embodiments, the compositions and methods of the invention are useful in treating anxiety diseases and disorders such as phobias, panic disorders, psychosocial stress (e.g. as seen in disaster, catastrophe or violence victims), obsessive-compulsive disorder, substance use/abuse disorders, addiction, mood disorders (including depression), chronic pain disorders, pervasive development disorders (e.g., autism and intractable aggressive disorder), generalized anxiety disorder and post-traumatic stress disorder (PTSD).

The present invention relates to compositions and methods for treating or preventing an anxiety-related disease or disorder, such as, but not limited to, PTSD and general anxiety disorders (GAD). The present invention is based, in part, upon the finding that the activity of the right basolateral amygdala (BLA) in particular the pattern of theta oscillations—after aversive stimuli may be unique and perhaps triggered at a lower threshold in disorders such as PTSD or GAD. Thus, the present invention relates in part to compositions and methods for detecting an altered pattern of theta oscillations as an indicator of PTSD or GAD, and further relates, in part, to a system for altering the pattern of theta oscillations as a method of treating or preventing an anxiety disease or disorder.

In one embodiment, the invention relates to a method of diagnosing a subject as having an anxiety related disease or disorder, the method comprising administering a stimulus to the subject, detecting at least one oscillatory pattern within the BLA that is consistent with an anxiety disorder and diagnosing the subject as having an anxiety related disease or disorder based on the detected oscillatory pattern. In one embodiment, the oscillatory pattern is a pattern of BLA gamma oscillations, a pattern of BLA theta oscillations or a theta-gamma coupling pattern. In one embodiment, the oscillatory pattern is an increase in BLA theta power. In one embodiment, the oscillatory pattern is an increase in BLA gamma power. In one embodiment, the oscillatory pattern is phase-amplitude coupling between BLA and another relevant brain region. In one embodiment, the relevant brain region is the hippocampus or OFC. In one embodiment, the oscillatory pattern is a signal detectable by current closed-loop neuromodulation system. In one embodiment, the stimulus is a negative neutral stimulus.

In one embodiment, the invention relates to a method of treating an anxiety related disease or disorder, the method comprising administering deep brain stimulation to a subject identified as having an anxiety related disease or disorder. In one embodiment, the deep brain stimulation alters at least one oscillatory pattern within the BLA. In one embodiment, the oscillatory pattern is a pattern of BLA gamma oscillations, a pattern of BLA theta oscillations or a theta-gamma coupling pattern. In one embodiment, the oscillatory pattern is an increase in BLA theta power. In one embodiment, the oscillatory pattern is an increase in BLA gamma power. In one embodiment, the oscillatory pattern is phase-amplitude coupling between BLA and another relevant brain region. In one embodiment, the relevant brain region is the hippocampus or OFC.

In one embodiment, the invention provides a method of diagnosing or treating an anxiety related disease or disorder using a detection-emission neuromodulation device. In this embodiment, electrodes are placed in the basolateral amygdala bilaterally and connected to a detection/emission device. Once the PTSD patient is subjected to a trauma reminder, an increase in power in the theta range occurs and is detected by the device. A pre-programmed electrical signal is produced by the device in order to abolish the establishment of the neural enhanced fear state in the basolateral amygdala.

### Definitions

Unless defined otherwise, all technical and scientific terms used herein have the same meaning as commonly understood by one of ordinary skill in the art to which this invention belongs. Any methods and materials similar or equivalent to those described herein can be used in the practice or testing of the present invention, exemplary methods and materials are described.

Generally, the nomenclature used herein and the laboratory procedures in organic chemistry are those well-known and commonly employed in the art.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

The term “bursts” as used herein means at least two spikes with an inter-spike interval of less than 10 ms.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a symptom of the disease or disorder, the frequency with which such a symptom is experienced by a patient, or both, is reduced.

An “effective amount” or “therapeutically effective amount” is that amount of a compound or electrical stimulation which is sufficient to provide a beneficial effect to the subject or mammal to which the compound or electrical stimulation is administered for a therapeutic treatment.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

A “therapeutic” treatment is a treatment administered to a subject who exhibits signs of pathology, for the purpose of diminishing or eliminating those signs.

As used herein, “treating a disease or disorder” means reducing the frequency with which a symptom of the disease or disorder is experienced by a patient. Disease and disorder are used interchangeably herein.

The term “recording site”, as used herein means a component or region of an electrode array that receives a potential electrical signal from an adjacent neuron for recordation.

The term “synchrony”, as used herein means a concurrence of an event between at least two or more things in a designated timeframe. For at least two neurons to be in synchrony, the at least two neurons may fire an electrical signal, or remain “silent”, in a concurring manner within a designated timeframe. Alternatively, synchrony can be between at least one neuron and the local field potential. In this case, the neuron fires an electrical signal or remains silent at a specific phase of an on-going oscillation in the local field potential. Alternatively, there could be synchrony between the local field potential recorded from two different microelectrodes. In this case, the phase of the oscillation in the local field potential recorded from one electrode concurs within a designated timeframe of a specific phase of the oscillation in the local field potential recorded from another electrode.

The term “neural spike”, as used herein means the recordation of an action potential from a single neuron. An action potential is an all-or-none change in membrane potential of the cell that is described by Hogkin and Huxely, 1950.

The term “moving average filter”, as used herein means a system in which the output is the mean of a shifting subset of inputs.

The term “Spike field coherence” (SFC), or “coherence”, as used herein refers to a measure of the strength of correlation between the spike times of a neuron or neuron population and the phase of the concurrent local field potential at any given frequency as described more completely by Grasse and Moxon, 2010.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention is based, in part, on the identification of theta oscillation patterns in the basolateral amygdala (BLA) that are associated with neurological and cognitive diseases and disorders. The present invention relates to compositions and methods for diagnosing and treating neurological and cognitive diseases and disorders. In various embodiments, the compositions and methods of the invention are useful in diagnosing or treating anxiety diseases and disorders such as phobias, panic disorders, psychosocial stress (e.g. as seen in disaster, catastrophe or violence victims), obsessive-compulsive disorder, substance use/abuse disorders, addiction, mood disorders (including depression), chronic pain disorders, pervasive development disorders (e.g., autism and intractable aggressive disorder), generalized anxiety disorder and post-traumatic stress disorder (PTSD).

The present invention relates to compositions and methods for treating or preventing an anxiety-related disease or disorder, such as, but not limited to, PTSD and general anxiety disorders (GAD). The present invention is based, in part, upon the finding that the activity of the right basolateral amygdala (BLA) in particular the pattern of theta oscillations—after aversive stimuli may be unique and perhaps triggered at a lower threshold in disorders such as PTSD or GAD. Thus, the present invention relates in part to compositions and methods for detecting an altered pattern of theta oscillations as an indicator of PTSD or GAD, and further relates, in part, to a system for altering the pattern of theta oscillations as a method of treating or preventing an anxiety disease or disorder.

In some embodiments, the invention relates to a closed-loop brain computer interface (BCI) system for treating neurological and cognitive diseases and disorders with responsive brain stimulation. In one embodiment, the system may be designed to record and stimulate brain circuits. For example, in one embodiment, an increase in BLA theta-power (3-12 Hz) following the presentation of a negative stimuli is an indicator of PTSD or GAD. In one embodiment, enhanced coupling between BLA gamma amplitude to particular phases of BLA theta oscillations following the presentation of a negative stimuli is an indicator of PTSD or GAD. In one embodiment, increased coupling between hippocampal gamma activity to a particular phase of BLA theta oscillations following the presentation of negative stimuli is an indicator of PTSD or GAD.

In one exemplary embodiment, the invention relates to an algorithm that may “decode” the patient's current capacity for fear extinction, from the BLA theta power, BLA gamma power, coupling between BLA gamma amplitude to particular phases of BLA theta oscillations, coupling between hippocampal gamma activity to a particular phase of BLA theta oscillations or coupling between BLA and other relevant brain regions such hippocampus and OFC.

Neural oscillations from various combinations of the brainwave frequency bands have been shown to exhibit coupling with one another, wherein one or more characteristics of one type of brainwave effect (or are affected by) one or more characteristics of another type of brainwave. In general, the coupling phenomenon is referred to as cross-frequency coupling. Combinations of frequency bands couple with one another to different degrees, while the coupling of various types of brainwaves may occur in connection with physiologic behavior or pathologic behavior. While certain cross-frequency coupling is exhibited in physiological or normal activity, other types of cross-frequency coupling may be developed in selected brain locations as a result of one or more neurological disorders. Also, cross-frequency coupling caused by one or more neurological disorders may occur intermittently.

For example, in one embodiment, coupling between BLA gamma amplitude to particular phases of BLA theta oscillations has been identified in connection PTSD or GAD. As another example, coupling between hippocampal gamma activity to a particular phase of BLA theta oscillations has been identified in connection with PTSD or GAD. In some embodiments the methods of the invention detect a persistent or intermittent pathological neural oscillation signature and provide neurostimulation to address the detected cross-frequency coupling to treat the one or more neurological disorders in a patient.

### Neurostimulation Systems

In some embodiments, the invention relates to a neurostimulation (NS) system that delivers stimulation therapies in accordance with embodiments herein. For example, the NS system may be adapted to stimulate spinal cord tissue, peripheral nervous tissue, deep brain tissue, or any other suitable nervous/brain tissue of interest within a patient's body.

Optionally, the NS system may represent a closed loop neurostimulation device that is configured to provide real-time sensing functions from a lead. The configuration of the lead sensing electrodes may be varied depending on the neuronal anatomy of the sensing site(s) of interest. The size and shape of electrodes is varied based on the implant location. The electronic components within the NS system can be configured for both stimulation and sensing. In one embodiment, the NS system includes an implantable medical device (IMD) that is adapted to generate electrical pulses for application to tissue of a patient.

The NS system may be programmed or controlled to deliver various types of stimulation therapy, such as tonic stimulation, high frequency stimulation, burst stimulation, noise stimulation, and nested stimulation therapies and the like. High frequency neurostimulation includes a continuous series of monophasic or biphasic pulses that are delivered at a predetermined frequency. Burst neurostimulation includes short sequences of monophasic or biphasic pulses, where each sequence is separated by a quiescent period. In general, nested therapies include a continuous, repeating or intermittent pulse sequence delivered at a frequency and amplitude with multiple frequency components.

The NS system may deliver stimulation therapy based on preprogrammed therapy parameters. The therapy parameters may include, among other things, pulse amplitude, pulse polarity, pulse width, pulse frequency, interpulse interval, inter burst interval, electrode combinations, firing delay and the like.

In one embodiment, suitable processing such as bandpass filtering may be applied using one or more analog and/or digital filters to separate neural activity associated with respective frequency bands. For example, neural activity may be processed to identify activity in respective neural activity bands (sigma, gamma, beta, alpha, theta, delta, infraslow bands).

In some embodiments, the instantaneous amplitude and/or phase in respective activity bands are determined. The instantaneous amplitude and/or phase in the respective activity bands may be determined using Fast Fourier Transform (FFT), Hilbert transforms, wavelet analysis, waveform-based estimation (via identification of intra-band waveform maxima, minima, and zero crossings), and other suitable techniques. Phase orthogonalization of signals may be employed before analyzing power envelope correlations (equivalently removing, after Fourier transformation, components of the same phase for the two respective signals).

In some embodiments, frequency relationships may be determined (such as cross-frequency coupling relationships). The determination of frequency relationships may include determining an amount or percentage of time that identified coupling activity with identified frequency characteristics have occurred. In one embodiment, a logical determination is made whether a pathological neural oscillation signature is detected. If so, in one embodiment, suitable neurostimulation is applied to one or more neural sites.

In one embodiment, nested stimulation may be applied to one or more neural network sites in response to detection of a pathological neural oscillation signature or activity. Nested stimulation may stimulate neuronal sites according to different types of neural oscillations. The different types of neural oscillation or brain wave activity can be decomposed into distinct frequency bands that are associated with particular physiologic and pathologic characteristics. In some embodiments, nested stimulation may be applied such that two, three or more frequency bands are coupled to one another to achieve various results.

Nested stimulation according to some embodiments applies multiple frequency bands to form a stimulation waveform or pattern. The waveform may be analog waveform. Alternatively, the stimulation pattern may include discontinuous pulses in other embodiments. The frequency bands may include respective bands from infraslow frequencies to sigma frequencies.

Brain Computer Interface (BCI) system

In one embodiment, the invention relates to an affective BCI component in a closed-loop, symptom to detect or alter one or more of a BLA theta oscillation, coupling between BLA gamma amplitude to particular phases of BLA theta oscillations, or coupling between hippocampal gamma activity to a particular phase of BLA theta oscillations. In one embodiment, the system is configured to diagnose patients by diagnostic assessment of objective measurement of the patient's performance on quantitative tests of psychological function. In one embodiment, the system is configured to link the diagnostic symptom assessment to a treatment that specifically activates or de-activates one or more brain areas via neurostimulation.

In one embodiment, the system is designed to record and decode neural information from specific dysfunctional networks associated with specific symptoms or behaviors and then deliver stimulation to these networks to afford symptom relief and measurable improvements in dysfunctional behaviors. In one embodiment, the patient may be given control over the stimulation system's actions through a hybrid BCI algorithm that monitors the patient's intentions. In one embodiment, the closed-loop algorithm further focuses the treatment not only in space (i.e., region of the brain) but in time, so that stimulation occurs only when the patient needs it.

In one embodiment, the system includes a central decoding and controlling hub and connected satellite modules that deliver stimulation and recording through electrodes. In some embodiments, the hub and satellite modules may be a single implanted module. Either part may also exist outside the body and communicate wirelessly to the electrodes. The hub, or implanted module, may be implanted under the scalp of a patient and can wirelessly communicate with an external base station for data streaming, reprogramming, wireless recharge, and coordinating intervention across sites to enhance treatment of neuropsychiatric dysfunction. Alternatively, the hub may itself be wearable or otherwise non-implanted, or the hub and base station may be merged as a single component. In one embodiment, the base station is in electrical communication with the hub via a head mounted interface and, in some embodiments, may interface with an offline processor having a user interface, such as a clinician interface. The head mounted interface may be a wearable processing unit that communicates, configures, and can control the implanted system. The head mounted interface might also mount or implant to some other body part (e.g., chest) depending on the surgical clinician's preference. In some embodiments, a hand held patient controller (e.g., a watch) may be provided for self-reporting and triggering recordings, as well as monitoring heart rate wirelessly, skin conductance, and the like.

In some embodiments, the system may be a non-invasive system for stimulating the brain. That non-invasive realization may collapse components such as the satellites, base station, and hub into a single part. The non-invasive system may enable not only treatment via induction of brain plasticity, but also pre surgical planning of DBS targets. The non-invasive system may be combined with EEG, for example, to produce a closed-loop system. The non-invasive system may include a plurality of scalp or non-contact electrodes and/or neuro-stimulation coils in communication with a software-controlled helmet, cap, or set of electrodes that can stimulate areas of the brain. The embedded software may steer the amount and polarity of energy sent to each electrode, thus shaping the E-fields and allowing accurate targeting of specific cortical areas. In other embodiments, the non-invasive system may be combined with non-electrical modalities, such as magnetic or ultrasonic stimulation, for stimulating the brain.

In some embodiments, algorithms stored on the hub may enable the system to merge spike and field-potential data to estimate the patient's psychiatric state and deliver therapeutic stimulation. The frequency of stimulation delivery may depend upon how frequently neural signatures that trigger stimulation occur. Those signatures may be fully or partly under a patient's direct intentional control. Real-time telemetry may enable a clinician, for example, to tune algorithm parameters as required by the patient. Thus, in some embodiments, the system providesreal-time brain activity in fully conscious patients interacting with real-world environments.

The system may be configured to operate in one or more modes. For example, in autonomous mode, the hub may be controlled by an internal processor and powered by an internal battery that can be recharged periodically. Low-bandwidth (e.g., 2 MBps) telemetry may be used to report on the hub's state of health and provide the subject comfort in the ability to both wake up and put the system to sleep when needed. In a continuous recharge mode, the head-mounted interface can be attached to the base station to wirelessly power the implanted device via a charger. The continuous recharge mode may be desirable when operating the system in modes that consume more power. In a base station control mode, a high-bandwidth telemetry link can be used to stream live neural data to be processed within the base station, which can then control stimulation therapies over the low-bandwidth telemetry. The base station control mode permits higher-power algorithms to be implemented without burden on the hub and satellite modules. In a computer control mode, the offline processor can be connected through the base station to further increase processing power and provide additional interfaces and resources to researchers and clinicians. The computer control mode may be used during the initial configuration period, where the implanted device may be running sub-optimally with high-channel counts and high processing power. As the device and algorithms are iteratively matched to the patient's needs, operation may become less dependent upon external modes and move toward autonomous control.

Each satellite module may interface with or be fully integrated into an electrode. The electrodes may be multi-channel macro-electrodes or micro-electrodes. A cross-point switch (CPS) matrix (Neural CPS) may be used to re-configure the electrodes for recording and stimulation. Additionally, the neural CPS may enable multiple electrodes to be simultaneously connected to one of two analog stimulus inputs to create lower impedance larger-area electrode clusters for stimulation. Neural amplification and digitization within the satellite module may provide a higher level of signal to noise ratio (SNR) and reduce the wire-count burden to communicate with the hub through multiplexed data. A field-programmable gate array (FPGA) may reduce wire-counts and provide control over the Neural CPS and amplifier. The FPGA may also provide charge-balanced communication with the hub, which mitigates risks of tissue damage caused by DC leakage currents. Power may be provided by an AC power supply that will convert an AC supply provided by the hub into DC voltages required by the satellite modules.

In one embodiment, the system records several types of neural signals (e.g., Spikes, LFPs, ECoG, etc.) from different types of electrodes (e.g., Micro-, DBS, ECoG arrays, etc.). In such an embodiment, the satellite may include a neural amplifier, such as a multi-purpose re-configurable neural amplifier that is both low-noise and low-power. Low noise may be necessary to capture small LFP and ECoG features in high gamma frequency bands, while low power may be necessary to reduce heat dissipation and extend the operational lifetime.

The hub may configure the satellite modules, process neural data, decode neuropsychiatric states, control closed-loop neurostimulation therapies, and transmit data to the external base station. The hub may include the processor, which may be a low-power processing core configured to execute algorithms. The hub may further include one or more current pulse generators for applying stimulation therapies through the electrodes. In addition, the hub may include a memory, such as dynamic random-access memory, accessible by the processor for stored data. The hub may also include the high-bandwidth telemetry link for neural data exfiltration, and the battery, which may be wirelessly charged, for extended operational life.

The current pulse generators for neural stimulation may be positioned in the hub and may be responsible for generating the programmable current-controlled stimulation pulses for neuromodulation. Analog stimulation waveforms may be sent to the satellite modules and routed to stimulation electrodes via the neural CPS. In order to effectively modulate stimulation therapies based upon closed-loop neural activity, stimulus waveforms may be dynamically re-programmable by hub algorithms. Waveforms may be biphasic and charge balanced, and voltages across the electrode-tissue interface may be limited to the water window to inhibit chronic tissue damage. For monopolar stimulation, currents may be returned to the conductive hub, and for bipolar stimulation, currents may return through adjacent electrodes.

The system may further include one or more sensors including, for example, humidity, temperature, and accelerometer sensors. Humidity and temperature sensors may be a useful part of health monitoring. Accelerometer data gathered may be useful for determining subject activity, including sleep, which may be used as a signal for closed-loop control.

The hub may include the processor and a control unit, such as a programmable logic controller (PLC) to manage system-level functions and execute closed-loop algorithms for adaptive neuromodulation therapy. The processor and control unit may be adaptable and re-programmable in order for closed-loop algorithms to be developed, tested, and tuned for enhanced therapeutic benefit to each patient. The processor and control unit may be capable of configuring satellite modules and receiving neural data, extracting signal features from raw neural data, decoding neuropsychiatric states, modulating stimulation therapy, monitoring and logging system health data, detecting and recording neural data, and managing wireless communication to the base station.

### Methods

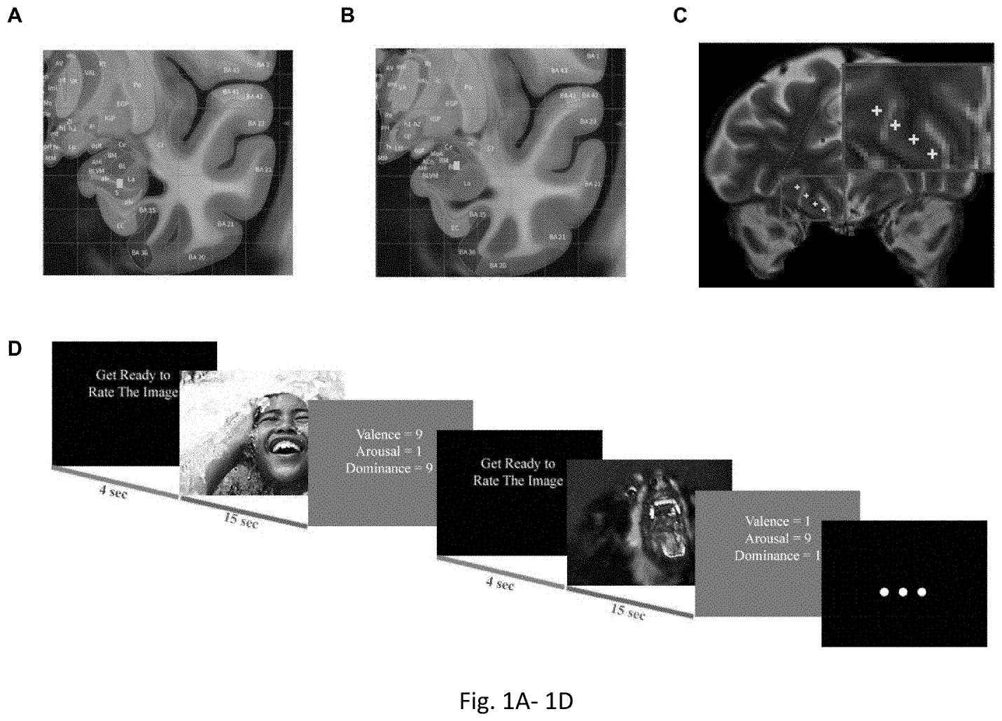

FIG. 1 provides a flow chart setting forth exemplary steps for diagnosing a subject as having a neurological or cognitive disease or disorder by analyzing an individual's brain with imaging testing. The method incorporates administering a stimulus to the subject, detecting at least one oscillatory pattern within the basolateral amygdala (BLA) that is consistent with an anxiety disorder and diagnosing the subject as having an anxiety related disease or disorder based on the detected oscillatory pattern.

In some embodiments, the method is a method of diagnosing a subject as having phobias, panic disorders, psychosocial stress (e.g. as seen in disaster, catastrophe or violence victims), obsessive-compulsive disorder, substance use/abuse disorders, addiction, mood disorders (including depression), chronic pain disorders, pervasive development disorders (e.g., autism and intractable aggressive disorder), generalized anxiety disorder or post-traumatic stress disorder (PTSD).

In general, the method may include performing a series of brain scans on a patient while the patient performs at least one behavioral or cognative task. From the patient's performance on the at least one task and/or the brain's activations during performance of the at least one task, the system may identify what is abnormal for that individual patient (e.g., a hyperarousal/hypervigilant state, fear, reward motivation, emotion regulation, decision making/impulsivity, attention/preservation, cognition, etc.). The system then may link the patient-specific behavioral measurement to patterns of activation and de-activation across different brain regions, identifying specific structures that are the source of the patient's individual impairment. The system may then use a range of invasive or non-invasive brain stimulation technologies to specifically target the brain regions and provide individualized psychiatric treatment, or to probe the network further to better classify the impairment.

The diagnostic assessment may be performed on a computer, for example, and may be administered as one or more simple computer games. In one embodiment, the diagnostic assessment may be administered to a patient who has a difficulty with fear regulation (a subset of difficulties with emotion regulation) which may commonly be found in a patient who has PTSD, but can also be seen in obsessive-compulsive disorder, generalized anxiety disorder, panic disorder, and sometimes major depressive disorder. Such a patient may have several psychiatric diagnoses, for example, a patient may be a returning service member who had been diagnosed with PTSD, but also with an alcohol use disorder and sub-threshold depressive symptoms. All of the below descriptions of task-based diagnostics and resulting stimulation protocols may be combined with the patient-intention controller described in below to form a hybrid brain-computer interface.

In one embodiment, during the associative learning task, the patient may be presented with a series of stimuli, such as a series of images, and be instructed to rate each image with respect to one or more parameters of valence (positive/negative), arousal (high/low), and dominance (high/low). While the patient is performing one or more task, brain activity, or neural oscillations, may be recorded simultaneously through non-invasive or invasive methodologies.

In one embodiment, the patient is asked to complete a virtual reality (VR) video game with a realistic and immersive environment. During the task, the patient may be presented with various stimuli (e.g., fear-provoking stimuli). While the patient is performing the task, neurophysiological activity and physiological markers are recorded in a synchronous manner. In some embodiments, the recorded signals are used as signals in a closed-loop system to then administer neurostimulation.

Diagnostic assessments of population norms may be acquired from a database of patients without evident psychiatric impairment who have performed the diagnostic tasks. The patient's performance on the diagnostic tasks may be compared to healthy controls by the system, or alternatively a trained clinician. As previously described, performance varies from task to task and may include, for example, how a subject rates specific stimuli.

Analysis and interpretation of a diagnostic assessment may classify patients by a distance from an origin. In one embodiment, the origin represents the population mean of healthy controls in a diagnostic space.

In one embodiment, imaging data from the patient may be compared to population averages to identify the brain regions where the patient has abnormally high or low levels of brain activity. Alternately, the population averages of other patients with similar behavioral performance may be substituted as a proxy for the individual patient's brain activity.

In some embodiments, the methods of the invention further include treating an anxiety related disease or disorder, the method comprising administering deep brain stimulation to a subject identified as having an anxiety related disease or disorder.

In some embodiments, regions or sub-regions are selected, based on the imaging data obtained, for stimulation using a neurostimulation device. Stimulation modalities for applying stimulation to the identified brain regions and sub-regions may include, but are not limited to, non-invasive electro-magnetic modalities (e.g., transcranial magnetic stimulation, transcranial direct- or alternative-current stimulation, transcranial focused ultrasound, infrared/optical through-skull modulation, etc.), invasive electro-magnetic modalities, and invasive optical modalities. In the case of invasive electro-magnetic modalities, electrodes or other amplifying devices may be surgically implanted into one or more brain regions and/or sub-regions. In the case of invasive optical modalities, transfection of one or more brain regions with proteins or other molecules may be involved that make neurons sensitive to light. Invasive optical modalities for stimulation may also involve implanting optical fibers into the brain regions. Combined non-invasive and invasive realizations, such as implantation of magnetic particles that then respond to applied magnetic fields, would also be reasonable.

Once stimulation is applied to the identified brain regions, intentional control over the neurostimulation system and over the application of a control algorithm may be provided to the patient. By providing patient intentional control, difficulties with over-control and emotional numbing, for example, may be circumvented. The closed-loop controller may be an emotional/affective brain-computer interface (BCI) that actively monitors the patient's emotional experience as expressed in the patient's brain activity.

In one embodiment, the stimulation may be adjusted in real-time to modulate the identified abnormal signals within the target brain regions. The stimulation may be directly adjusted using, for example, real-time recordings of brain electrical signals for closed loop control. In one non-limiting example, if the patient receives an invasive stimulation modality including implanted electrodes, and a neuro-stimulation pulse generator, to deliver electricity, stimulation may be delivered in a closed loop fashion. That is, the system may directly monitor the brain's electrical activity at the implant sites, and may alter the stimulation dose at each site based on observations throughout the network. In some embodiments, monitoring electrical activity may be done optically, for example, through genetically encoded voltage reporters. Monitoring may attempt to directly infer the patient's emotional state, to respond to the patient's intentional commands, or to merge both types of monitoring into a hybrid system.

In order to adequately adjust stimulation in the various brain regions, an emotional decoding algorithm may be stored on the controlling hub of the system that can infer the patient's current emotional/symptom state from brain activity. The emotional decoding algorithm may utilize the time-resolved imaging and recording data acquired. For example, in one embodiment, an increase in BLA theta-power (3-12 Hz) is an indicator of PTSD or GAD and therefore, in one embodiment, the emotional decoding algorithm may decode the patient's current state from relative theta-power in the BLA. In one embodiment, enhanced coupling between BLA gamma amplitude to particular phases of BLA theta oscillations following the presentation of a negative stimuli is an indicator of PTSD or GAD, and therefore, in one embodiment, the emotional decoding algorithm may decode the patient's current state from BLA gamma amplitude and BLA theta oscillations. In one embodiment, increased coupling between hippocampal gamma activity to a particular phase of BLA theta oscillations following the presentation of negative stimuli is an indicator of PTSD or GAD therefore, in one embodiment, the emotional decoding algorithm may decode the patient's current state from hippocampal gamma activity and BLA theta oscillations.

In some embodiments, the emotional decoding algorithm may utilize artificial intelligence, machine learning or other strategies to adjust over time. In some embodiments, the emotional decoding algorithm adjusts itself over time to improve the accuracy and sensitivity of the detections.

Regardless of the method used to adjust stimulation, in one embodiment, there is a signal that the controller hub is programmed to sense. The controller hub may then adjust stimulation (e.g., the stimulation intensity or different parameters) until the signal returns to a pre-defined range. For the hippocampal or amygdala activity may be directly sensed, and the controller hub may be configured to keep the amygdala activity within the pre-defined range (e.g., a pre-measured, non-anxious baseline). When amygdala activity exceeded the pre-defined range, the controller hub may be configured to interpret this as the patient experiencing a hypervigilance state, and would drive the implanted neurostimulator to shut down amygdala activity.

In one embodiment, the system may use the patient's brain signals as a read-out of what he/she wants the stimulator to be doing, then use that to guide application of the closed-loop system. This may be called a hybrid BCI because there is an autonomous part (e.g., the emotional decoder) and a patient-controllable part (the intention decoder), and the two are coupled together to achieve adequate clinical performance. In some realizations, the emotional decoder may not be needed, or may be relatively trivial (e.g., the monitoring of a single channel in a single brain area).

It is to be understood that the subject matter described herein is not limited in its application to the details of construction and the arrangement of components set forth in the description herein. The subject matter described herein is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore are not to be construed as limiting in any way the remainder of the disclosure.

### Example 1: Intracranial Neurophysiology of Hypervigilance in Posttraumatic Stress Disorder

Here, how oscillatory interactions in specific brain regions may coordinate enhanced fear and hypervigilance in PTSD is investigated. Local field potentials (LFP) were directly recorded from the BLA (FIG. 1A), anterior HPC (FIG. 1B), and OFC (FIG. 1C) in a patient with PTSD. Without being bound by theory, it was hypothesized that: (i) there is an increase in the BLA theta power following the presentation of a negative compared to positive/neutral stimuli, (ii) there is enhanced coupling between BLA gamma amplitude to particular phases of BLA theta oscillations following the presentation of a negative stimuli, and (iii) there is increased coupling between hippocampal gamma activity to a particular phase of BLA theta oscillations following the presentation of negative stimuli.

In order to examine these, the effects of positive/neutral and aversive images on BLA, HPC, and OFC LFP activity were examined in a patient suffering from severe treatment-refractory PTSD. The behavioral task and invasive recordings (Nihon Koden; sampled at 10 KHz) were completed intraoperatively at the time of deep brain stimulation (DB S) electrodes placement prior to DB S in the context of ongoing DB S in PTSD clinical trial (Langevin et al. 2016, Brain Sciences 6:28). The behavioral task consisted of 30 images, selected from the international affect picture system (IAPS) that were displayed serially on a computer screen to the participant. After each image presentation (positive, negative, or neutral), the participant was immediately asked to rate the image on a scale from 1-9 of valence (positive/negative), arousal (high/low), and dominance (high/low) (FIG. 1D).

Images were divided into two categories: negative stimuli were defined as images with low (<5) valence and dominance scores, and high (>5) arousal score; the remainder of the images were categorized as positive/neutral (hereinafter referred to as positive). Accordingly, electrophysiological data was divided for positive and negative images for further analysis and negative images were interpreted as indicating a hyperarousal/hypervigilant state.

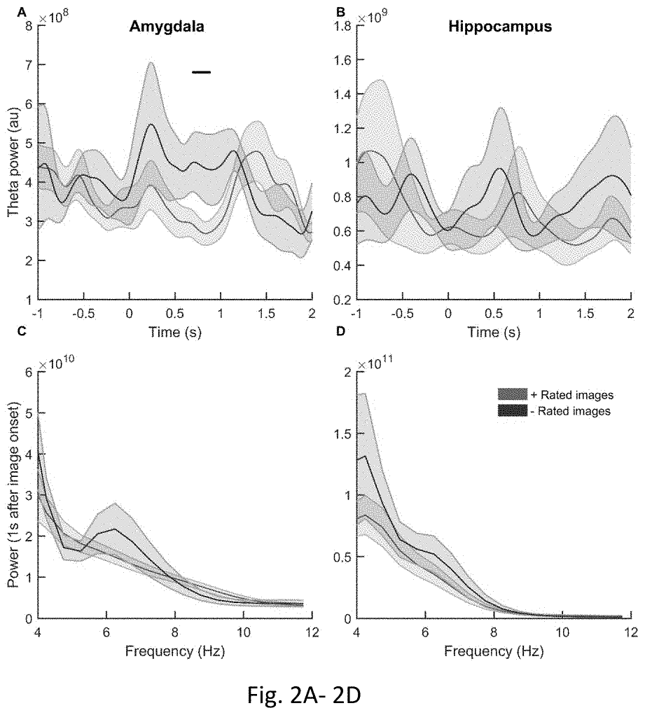

Whether there was a change in theta (3-12 Hz) power was evaluated in response to the presented images [prediction i]. To achieve this, a time-frequency analysis (Hughes et al., 2012, Hippocampus, 22:1417-1428) was performed and the theta power was calculated in the signal recorded from the contact localized to the right BLA around the image onset time, for negative and positive images separately. A statistically significant difference in BLA theta power was found after the onset of negative versus positive images, such that theta power was higher approximately 0.70-0.88s after the image onset (FIG. 2A). It is worth noting that this time frame is reminiscent of the window during which a sustained amygdala event related potentials (ERPs) are observed in epilepsy patients exposed to fearful facial expressions (Krolak-Salmon et al. 2004, Neuron, 42: 665-676). The calculated power was collapsed in the time domain (limited to 1 second after image onset), and it was observed that the difference in power between positive and negative stimuli is localized to theta frequencies around 6 Hz (FIG. 2C), consistent with previously reported results in rodents.

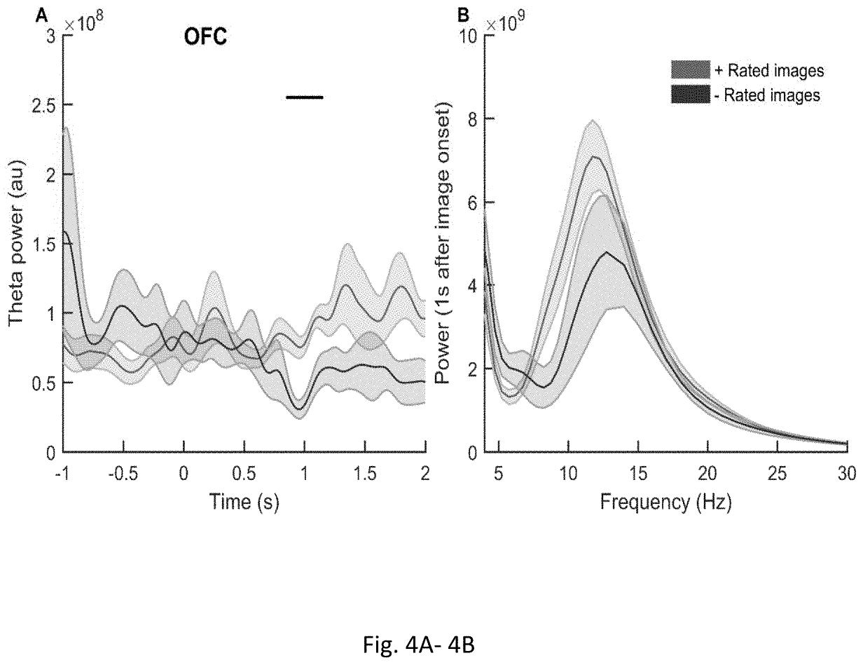

To determine whether this effect was specific to the BLA, the same analysis was performed on signals recorded from the right HPC as a control, and did not observe any effects (FIG. 2B, FIG. 2D). In contrast, theta power in the OFC showed a significant reduction around 0.86-1.14s after the presentation of negative stimuli compared to positive stimuli (FIG. 4). This effect may bear a resemblance to the reduction of OFC activity in fMRI studies (Williams et al., 2006, NeuroImage 29: 347-357). Thus, these findings suggest that BLA theta power, after aversive stimulus, may represent a reliable signature—found in humans and across species—signaling the onset of a fear-driven hypervigilant state.

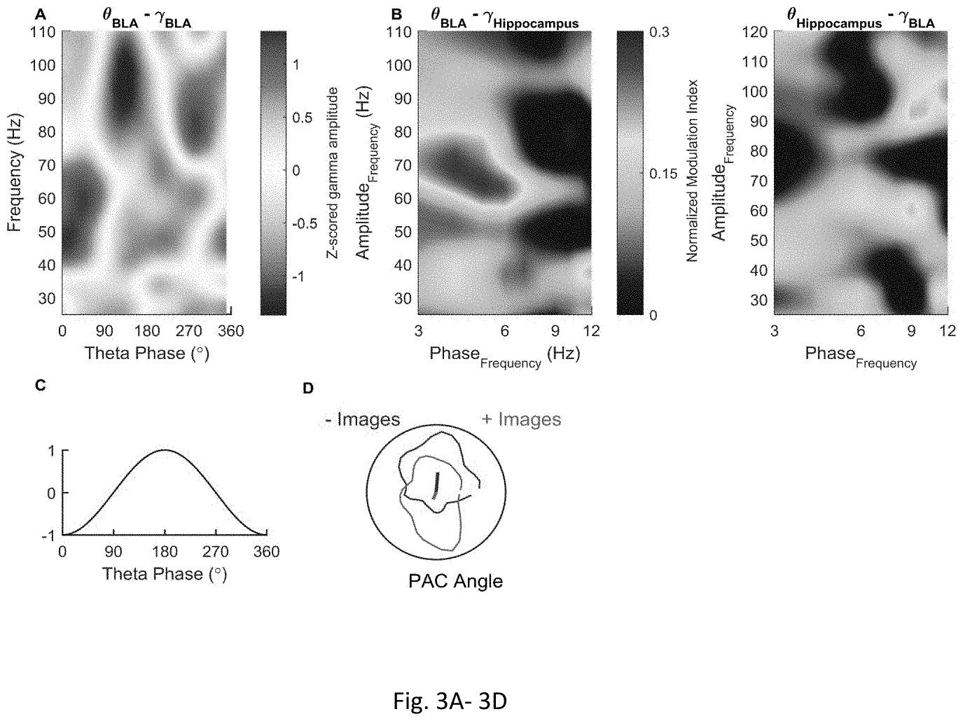

Furthermore, different patterns of cross-frequency coupling were explored within the BLA as well as between BLA and HPC. First, the nature of theta-gamma coupling within the BLA was explored by computing phase-amplitude co-modulogram using previously described methods (Belluscio et al., 2012, The Journal of Neuroscience: The Official Journal of the Society for Neuroscience, 32: 423-435). The data seem to suggest that low and high gamma oscillations are locked to different phases of theta for negative stimuli (FIG. 3A; prediction ii)—an observation that is consistent with results from rodent studies (Stujenske et al. 2014, Neuron, 83: 919-933).

Modulation of the HPC by the BLA provides further insight into the influence of fear on memories (Zheng et al. 2017, Nature Communications, 8:14413; Seidenbecher et al., 2003, Science, 301: 846-850). Across species and sensory modalities, the organization of oscillations tend to be consistent with the phase of lower frequency modulating the amplitude of higher frequency oscillations (Lakatos et al., 2005, Journal of Neurophysiology, 94:1904-1911). PAC between theta oscillations in the BLA and gamma oscillations in the HPC (Canolty and Knight, 2010, Trends in Cognitive Sciences, 14: 506-515; Tort et al. 2009, Proceedings of the National Academy of Sciences, 106: 20942-20947) were characterized. This characterization was done in two ways: (1) modulation index (MI) was calculated as a measure for the strength of theta-gamma coupling between BLA and HPC (FIG. 3B); (2) the preferred phase of modulation was computed to investigate whether the coupling occurs preferentially at certain theta phases (FIG. 3D). These results demonstrated that there was a directional theta-gamma coupling between the BLA and HPC, such that for negative stimuli, BLA theta was coupled to gamma oscillations in the HPC, while there was no coupling in the opposite direction (HPC theta to BLA gamma) (FIG. 3B). Furthermore, this coupling preferentially occurred at the rising phase of BLA theta (FIG. 3D; circular V-test, p<1⁻³; prediction iii). These results may suggest that the BLA influences memory encoding and/or retrieval by the HPC while a hypervigilance state is established. Memories under this state have been described as gist-based with poor detail encoding leading to a propensity for generalization and false recalling (Hayes, VanElzakker, and Shin, 2012, Frontiers in Integrative Neuroscience 6: 1-14).

Overall, the findings demonstrate that the BLA neuronal ensemble orchestrates states of hyperarousal and hypervigilance using a theta oscillatory pattern. Electrophysiologically, this could be characterized by a dominant BLA theta rhythm entraining gamma oscillations within the BLA and the HPC to coordinate alertness and gist-based recollection of upcoming aversive events. An increase in BLA theta oscillation power is recorded at the onset of the phenomenon therefore potentially signaling the imminent establishment of hypervigilant state. These findings are clinically relevant since the increase in BLA theta power is a signal detectable by current closed-loop neuromodulation systems (e.g., Neuropace responsive neurostimulator). Such a system would be able to detect and record the signal linked to hypervigilance and then emit an electrical response to disrupt the BLA theta PAC. In addition, the occurrence of the signal would be trackable over time to determine the fluctuation of PTSD symptoms severity with a higher frequency predicting periods of exacerbation.

Materials and Methods

Subject

The subject was a 40 years old veteran who fought in the US Marines as infantry during the Operation Iraqi Freedom conflict. Two events were especially traumatic for the patient. In the first incident, he witnessed his lieutenant being killed in a car explosion, moments after having a conversation with her. In a second event, his car was blown up by an improvised explosive device leaving him unconscious and injured. He required intense treatment and rehabilitation for his injuries and was subsequently diagnosed with post-traumatic stress disorder (PTSD). His primary symptoms are hypervigilance and hyperarousal with intense irritability. He avoids social interactions because he is afraid to lose control and get into a violent altercation.

Surgery

The patient underwent the placement of cranial fiducials (Waypoint, FHC) approximately two weeks prior to the surgery. The electrodes trajectories were planned using a stereotactic software (Navigator, FHC) and the pre-operative MRI. A trans-frontal trajectory was used and contacts are placed in the BLA and in the anterior HPC. Once the MRI is oriented along the AC/PC plane, the BLA is located anterior to the tip of the temporal horns and dorsal to the HPC. On the day of surgery, the custom stereotactic platform (Starfix, FHC) was secured to the fiducial markers. Linear incisions were made bilaterally anterior to coronal suture and small craniotomy openings were created with a high-speed drill along the trajectory defined by the Starfix platform (FHC). The dura was opened and a cannula was inserted along the pre-determined trajectory. The patient was fully awoken. A 10-contact recording electrode with platinum contacts measuring 2.4 mm each with 5 mm, center to center, spacing (Spencer probe, Adtech) was inserted into the cannula and the cannula was pulled back to ensure that all the electrode contacts were exposed.

Behavioral Task and Electrophysiological Recordings

Once fully awake, the patient was presented with a set of 30 images from international affect picture system (IAPS). The images were chosen from a diverse set of situations and were meant to elicit different emotions (positive, neutral and negative). The negative images were selected to have some war imagery component to serve as a trauma reminder. The subject was presented with each picture on a computer screen for 15 seconds and asked to rate the image on scale from 1-9 of valence (positive/negative), arousal (high/low), and dominance (high/low). Each electrode contact was connected to the electroencephalogram amplifier (Nihon Kohden); one of the dorsal contacts was used as reference. Electrophysiological data was recorded at 10 KHz, saved and exported as European Data Format for further analysis with Matlab. At the conclusion of the behavioral task, the recording electrode was exchanged for the permanent deep brain stimulation (DBS) electrode (3387, Medtronic) within the cannula. Post-operatively, a stereotactic CT scan was obtained and fused to the pre-operative MM to confirm the proper placement of the DBS and the recording electrodes along the intended trajectory.

Data Analysis

Power Analysis:

In order to study the power spectral analysis, the recorded LFPs were temporally aligned to the stimulus onset times (e.g., image onset). The BOSC toolbox was used to calculate power and a 6^{th} order wavelet was utilized.

Theta-Gamma Comodulograms within the BLA:

A finite impulse response (FIR) filter was applied to extract theta oscillations (3-12 Hz), and theta phase was computed as the angle of the Hilbert transformation of the signal. To extract gamma oscillations, the signal was filtered with a band pass FIR filter (width=4 Hz) at all frequencies above 20 Hz, in increments of 2 Hz. Theta phase was binned in 36 bins and within each bin, the amplitude of gamma oscillations was calculated.

Phase Amplitude Coupling Between the BLA and the HIP/OFC:

To create power coherence comodulograms, first, a set of band-pass filtered signals was created by filtering the LFP signal with an FIR filter centered at all frequencies above 2 Hz: for F_{g}=3-12 Hz in 2 Hz increments, with ±1 Hz surrounding each increment (bandpass width=2 Hz), and for F_{y}=25-125 Hz in 4 Hz increments, with ±2 Hz surrounding each increment (bandpass width=4 Hz). This was then followed by normalizing each bandpass by subtracting the mean and dividing by standard deviation of entire record (z-score normalization). The Modulation Index (MI) was calculated between the phase component of the low-frequency oscillation (theta) band Φ_{θ}, and the amplitude component of the high-frequency (gamma) band A_{γhigh} after stimulus/trial onset or stimulation offset with a method previously described. For example, to determine MI for N trials of particular condition (e.g., positive/negative images, CS+/−, high/low arousal, high/low dominance, recalled/missed), a trial-by-time matrix pre-filtered at the theta (3-12 Hz) range, {X_{θ}[t]}_{1:N}, was truncated to time regions [to: tend] corresponding to the time window of the trial, re-shaped into a 1-by-N·[t_{end}−t₀] vector of concatenated trials, and then converted into a complex valued signal with the Hilbert transform. The phase angle time series of this complex-valued signal results in the instantaneous phase of the low-frequency band, Φ_{θ}. Similarly, LFP trials pre-filtered at the high-frequency (e.g. high- or low-gamma) (>25 Hz) range {X_{γhigh}[t]}_{1:N} were truncated to time regions [t₀:t_{end}], vectorized, and converted via the Hilbert transform to complex-valued form; the complex modulus (absolute value) was used to compute the instantaneous high-frequency amplitude signal A_{γhigh}. To measure strength of coupling between the theta phase Φ_{θ} and gamma amplitude A_{γhigh}, those two signals were combined into an analytic complex-valued signal via Euler's formula, and then the resultant vector was computed (Vᵣₑₛᵤₗₜₐₙₜ), with the raw modulation index measured as the resultant vector length, while the preferred phase was measured as the resultant vector's angle:

$${V}_{resultant} = {\sum }_{t = 1}^{n} {A}_{γ high} \cdot {e}^{i {Φ}_{θ}} , {MI}_{raw} = \vert {V}_{resultant} \vert , \\ {MI}_{angle} = {tan}^{- 1} ( \frac{Im ( {V}_{resultant} )}{Re ( {V}_{resultant} )} )$$

Statistics:

For the analysis of PAC angles, circular statistics toolbox was used.

### Example 2: Similar Patterns of Theta Power in PTSD and GAD but not in Epilepsy

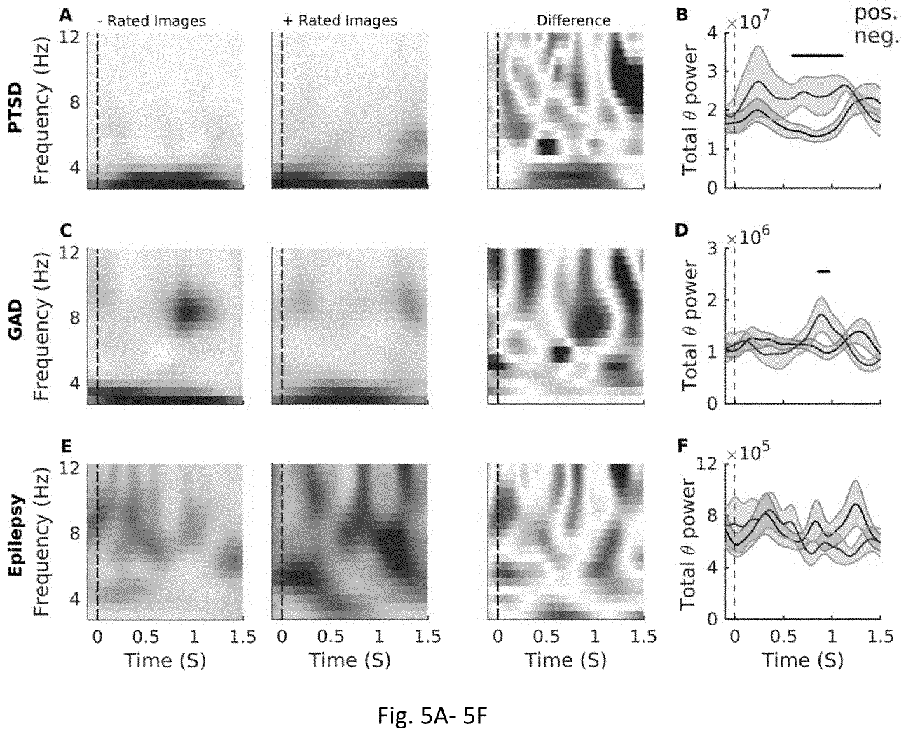

The results from Example 1, higher theta power in the right BLA after negative images, were replicated in a participant diagnosed with severe GAD. However, in a participant with epilepsy (who did not suffer from PTSD or GAD), no significant differences were observed in the BLA theta power after the negative versus positive/neutral stimuli (FIG. 5).

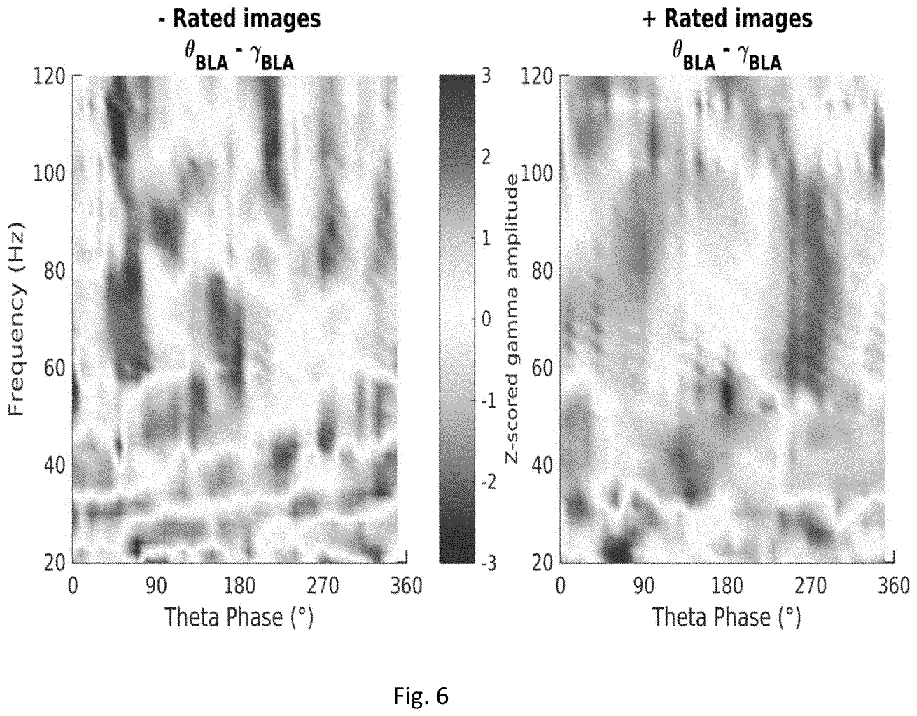

The nature of theta-gamma coupling was explored within the BLA by computing the phase-amplitude co-modulogram. The data (FIG. 6) suggests that low and high gamma oscillations are locked to different phases of theta for negative stimuli.

The results suggest that the activity of the right BLA—in particular the pattern of theta oscillations—after aversive stimuli may be unique and perhaps triggered at a lower threshold in disorders such as PTSD or GAD such that it is conducive to the coordination of an enhanced fear and a hypervigilant state.

BLA theta also significantly interacted with BLA gamma oscillations further corroborating the framework that the regulation of emotional states is mediated by specific oscillatory patterns within the BLA.

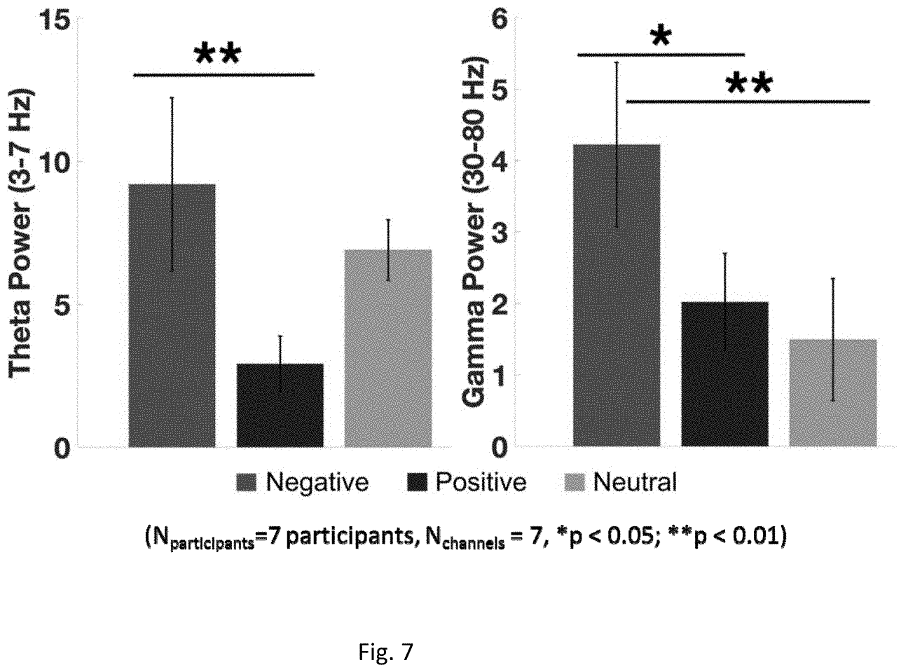

FIG. 7 includes the activity in the amygdala for 7 patients who are presented with the images (negative, positive and neutral). An increase in power in the theta range and the gamma range is seen for negative images compared to positive images.

These results, combined with the use of deep brain stimulation, may be clinically relevant in developing future therapy options for PTSD.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method of diagnosing a subject as having an anxiety related disease or disorder, the method comprising administering a stimulus to the subject, detecting at least one oscillatory pattern within the basolateral amygdala (BLA) that is consistent with an anxiety disorder and diagnosing the subject as having an anxiety related disease or disorder based on the detected oscillatory pattern.

2. The method of claim 1, wherein the oscillatory pattern is selected from the group consisting of a pattern of BLA gamma oscillations, a pattern of BLA theta oscillations and a theta-gamma coupling pattern.

3. The method of claim 1, wherein the oscillatory pattern is an increase in BLA theta power.

4. The method of claim 1, wherein the oscillatory pattern is an increase in BLA gamma power.

5. The method of claim 1, wherein the oscillatory pattern is phase-amplitude coupling between BLA and another relevant brain region.

6. The method of claim 5, wherein the relevant brain region is selected from the group consisting of the hippocampus and the orbitofrontal cortex (OFC).

7. The method of claim 1, wherein the oscillatory pattern is a signal detectable by a current closed-loop neuromodulation system.

8. The method of claim 1, wherein the stimulus is a negative neutral stimulus.

9. The method of claim 1, wherein the anxiety disease or disorder is selected from the group consisting of phobias, panic disorders, psychosocial stress (e.g. as seen in disaster, catastrophe or violence victims), obsessive-compulsive disorder, substance use/abuse disorders, addiction, mood disorders (including depression), chronic pain disorders, pervasive development disorders (e.g., autism and intractable aggressive disorder), generalized anxiety disorder and post-traumatic stress disorder (PTSD).

10. A method of treating an anxiety related disease or disorder, the method comprising administering brain stimulation to a subject identified as having an anxiety related disease or disorder.

11. The method of claim 10, wherein the brain stimulation alters at least one oscillatory pattern within the BLA.

12. The method of claim 11, wherein the oscillatory pattern is selected from the group consisting of a pattern of BLA gamma oscillations and a pattern of BLA theta oscillations and a theta-gamma coupling pattern.

13. The method of claim 12, wherein the oscillatory pattern is an increase in BLA theta power.

14. The method of claim 12, wherein the oscillatory pattern is an increase in BLA gamma power.

15. The method of claim 12, wherein the oscillatory pattern is phase-amplitude coupling between BLA and another relevant brain region.

16. The method of claim 15, wherein the relevant brain region is selected from the group consisting of the hippocampus and the orbitofrontal cortex (OFC).

17. The method of claim 10, wherein the brain stimulation is administered using a neuro-stimulation device.

18. The method of claim 17, wherein the neuro-stimulation device comprises an implanted module.

19. The method of claim 10, wherein the brain stimulation is deep brain stimulation.

20. The method of claim 10 wherein the anxiety disease or disorder is selected from the group consisting of phobias, panic disorders, psychosocial stress (e.g. as seen in disaster, catastrophe or violence victims), obsessive-compulsive disorder, substance use/abuse disorders, addiction, mood disorders (including depression), chronic pain disorders, pervasive development disorders (e.g., autism and intractable aggressive disorder), generalized anxiety disorder and post-traumatic stress disorder (PTSD).
